(19) United States
(12) Patent Application Publication
(10) Pub. No.: US 2011/0257537 A1
(43) Pub. Date: Oct. 20, 2011

(54) BLOOD PRESSURE MONITORING SYSTEM PROVIDING ENHANCED VASCULAR ANEURYSM DETECTION FEATURES AND RELATED METHODS

(76) Inventor: Anthony ALATRISTE, Windermere, FL (US)

(21) Appl. No.: 13/087,684

(22) Filed: Apr. 15, 2011

Related U.S. Application Data
(60) Provisional application No. 61/324,527, filed on Apr. 15, 2010.

(51) Int. Cl. A61B 5/021

(52) U.S. Cl. 600/485

(57) ABSTRACT

A blood pressure monitoring system may include a plurality of a blood pressure measuring devices each configured to measure blood pressure of a respective extremity of a patient, and a blood pressure monitoring device. The blood pressure monitoring device may include at least one interface device configured to interface with the plurality of blood pressure measuring devices, and a controller coupled to the at least one interface device. The controller may be configured to cooperate with the plurality of blood pressure measuring devices to determine respective blood pressure measurements for each patient extremity, selectively determine differences between the blood pressure measurements, and generate an indication of a probable vascular aneurysm based upon a determined difference between at least two of the blood pressure measurements reaching a vascular aneurysm threshold value.

### CROSS-REFERENCE TO RELATED APPLICATIONS

This application claims the benefit of provisional application no. 61/324,527, filed Apr. 15, 2010, the entire contents of which are hereby incorporated herein by reference.

### FIELD OF THE INVENTION

The present invention relates to the field of medical devices, and, more particularly, to blood pressure monitoring systems, devices, and related methods.

### BACKGROUND OF THE INVENTION

Various approaches have been developed for determining blood pressures of patients. One such example is disclosed in U.S. Pat. No. 7,608,045 to Mills, which is directed to a method for noninvasively monitoring physiological characteristics of a patient's blood. Determinations of blood constituent concentrations may be made by comparing absorbance of radiation at varying parameters, such as path length and blood pressure. Changes in pressure may be effected by changing the height of the probes relative to the patient's heart. Determinations of blood pH may be made by comparing absorbance of the blood at different wavelengths. The temperature of the blood, and thus of the patient's core, may also be accurately determined. Further, cardiac output characteristics and blood pressures may be noninvasively determined using the methods of the invention.

A related ankle brachial pressure index system is disclosed in U.S. pub. no. 2009/0036786 to Gough et al. The system includes two cuffs for each ankle and two cuffs for each arm of a patient. Each cuff has chambers. The four cuffs are applied to each limb (or finger or toe), and each chamber is inflated simultaneously to a pressure until a Pneumo Arterial Plethysmography (PAPG) signal related to the arterial flow in the limb is detected at the chambers. The chambers are then simultaneously inflated until the PAPG signals are extinguished in each limb, the inflation of chambers. continuing for 10 mmHg to 20 mmHg above that pressure. The chambers are then deflated and the pressure at which the PAPG signal returns in the first chamber is recorded for each limb and this value of the pressure is used to calculate the ABPI. The ABPI is displayed or sent to a remote site.

Despite the advantages of such systems, further enhancements may be desirable in blood pressure monitoring systems, such as to provide enhance diagnostic capabilities, for example.

### SUMMARY OF THE INVENTION

A blood pressure monitoring system is provided herein which may include a plurality of a blood pressure measuring devices each configured to measure blood pressure of a respective extremity of a patient, and a blood pressure monitoring device. The blood pressure monitoring device may include at least one interface device configured to interface with the plurality of blood pressure measuring devices, and a controller coupled to the at least one interface device. The controller may be configured to cooperate with the plurality of blood pressure measuring devices to determine respective blood pressure measurements for each patient extremity, selectively determine differences between the blood pressure measurements, and generate an indication of a probable vascular aneurism based upon a determined difference between at least two of the blood pressure measurements reaching a vascular aneurism threshold value. Accordingly, the system may advantageously be used by emergency responders or triage providers to quickly and accurately assess the potential of a vascular aneurism in a patient, and thereby potentially expedite obtaining appropriate follow-up treatment in time to help avoid serious injury or death.

More particularly, the blood pressure monitoring device may be further configured to determine a probable vascular aneurism type from among a plurality of different vascular aneurism types (e.g., thoracic aortic aneurysm, abdominal aortic aneurysm, peripheral arterial aneurysm, etc.) based upon the extremities associated with the at least two blood pressure measurements. Moreover, the blood pressure monitoring device may further include an input device coupled to the controller, and the controller may be further configured to receive an indicator of a localized pain region via the input device, and determine the vascular aneurism type also based upon the localized pain region indicator.

Additionally, the controller may be configured to cooperate with the plurality of blood pressure measuring devices to determine respective blood pressure measurements for each patient extremity on a staggered time schedule. Also, the controller may be further configured to determine at least one ankle brachial pressure index (ABI) value based upon the blood pressure measurements, and generate the indication of a probable vascular aneurism also based upon the at least one ABI.

The blood pressure monitoring device may further comprise at least one feedback device coupled to the controller. As such, the controller may be further configured to selectively output, via the at least one feedback device, a warning not to administer an anticoagulant to the patient based upon the determined difference between the at least two of the blood pressure measurements reaching the vascular aneurism threshold value. By way of example, the vascular aneurism threshold may comprise at least a twenty mmHg systolic pressure difference.

In addition, the blood pressure monitoring device may further comprise at least one display coupled to the controller, and the controller may be further configured to cause the at least one display to display a visual indication of the probable vascular aneurism. Moreover, the controller may be further configured to cause the at least one display to display the blood pressure measurements for each of the extremities. In some exemplary embodiments, at least one of the blood pressure measuring devices may comprise a wireless blood pressure measuring device, and the at least one interface device may comprise a wireless communications circuit.

A related blood pressure monitoring device, such as the one described briefly above, and blood pressure monitoring method are also provided. The method may include coupling a plurality of blood pressure measuring devices each to a respective extremity of a patient. The method may also include using a blood pressure monitoring device to cooperate with the plurality of blood pressure measuring devices to determine respective blood pressure measurements for each patient extremity, selectively determine differences between the blood pressure measurements, and generate an indication of a probable vascular aneurism based upon a determined difference between at least two of the blood pressure measurements reaching a vascular aneurism threshold value.

A related non-transitory computer-readable medium is also provided, which may have computer-executable instructions for causing a computing device to perform steps comprising: cooperating with a plurality of blood pressure measuring devices, each coupled to a respective extremity of a patient, to determine respective blood pressure measurements for each patient extremity; selectively determining differences between the blood pressure measurements; and generating an indication of a probable vascular aneurysm based upon a determined difference between at least two of the blood pressure measurements reaching a vascular aneurysm threshold value.

### BRIEF DESCRIPTION OF THE DRAWINGS

FIG. 1 is schematic block diagram of a blood pressure monitoring system in accordance with an exemplary embodiment.

FIG. 2 is a schematic block diagram of the blood pressure monitoring device of the system of FIG. 1 illustrating additional optional features thereof.

FIG. 3 is a front view of an example feedback device of the blood pressure monitoring device of FIG. 2.

FIG. 4 is a perspective view of another example embodiment of the blood pressure monitoring system of FIG. 1.

FIG. 5 is a flow diagram illustrating method aspects associated with the systems and devices of FIGS. 1-4.

### DETAILED DESCRIPTION OF THE PREFERRED EMBODIMENTS

The present invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

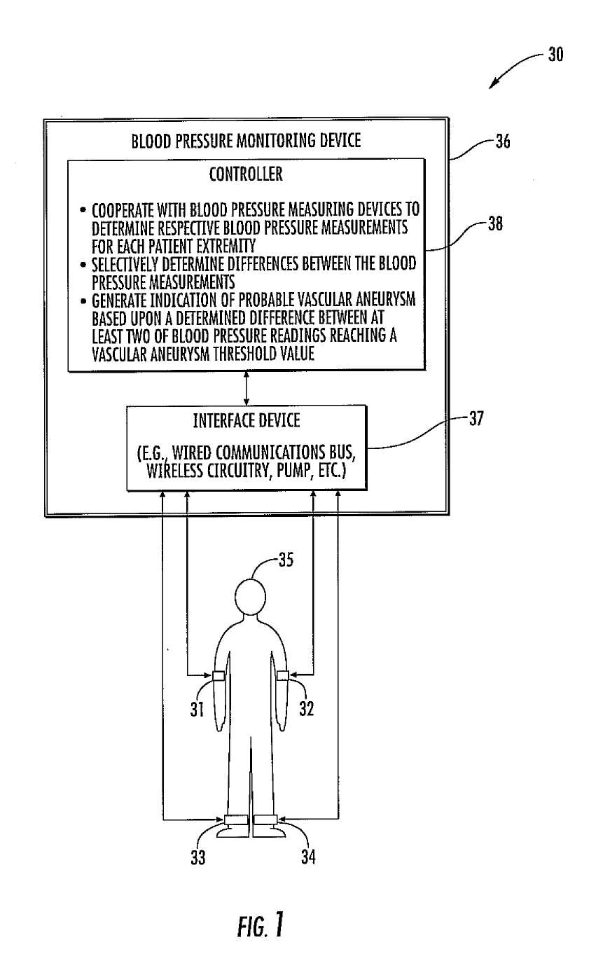

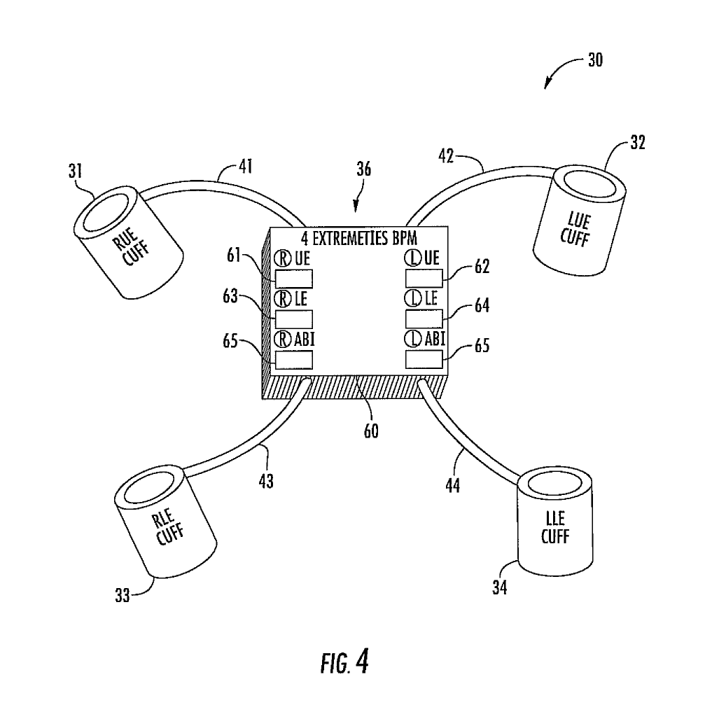

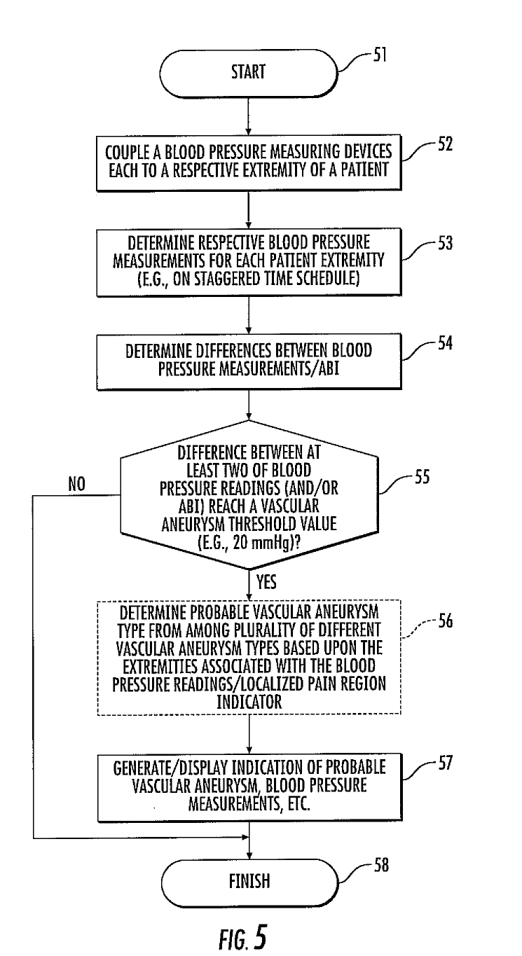

Referring now to FIGS. 1 through 5, a blood pressure monitoring system 30 and related method aspects are first described. The system 30 illustratively includes a plurality of a blood pressure measuring devices 31-34, each of which is configured to measure the blood pressure of a respective extremity of a patient 35. Beginning at Block 51 (FIG. 5), in the illustrated example, the blood pressure measuring devices 31-34 are respectively associated with or coupled to (Block 52) the following extremities: right upper extremity (RUE) or right arm; left upper extremity (LUE) or left arm; right lower extremity (RLE) or right leg; and left lower extremity (LLE) or left leg. In the example of FIG. 4, the blood pressure measuring devices 31-34 are cuffs, but other suitable types of blood pressure measurement devices (e.g., finger/toe probes, etc.) may also be used. Moreover, in the case of blood pressure cuffs, these may be reusable cuffs or disposable cuffs (or a combination thereof).

The system 30 further illustratively includes a blood pressure monitoring device 36. More specifically, the blood pressure monitoring device 36 illustratively includes one or more interface devices 37 configured to interface with the blood pressure measuring devices 31-34, and a controller 38 coupled to the interface device. By way of example, in some embodiments where blood pressure cuffs are used, the interface device 37 may comprise one or more air pumps or compressors (not shown) with attachments for fluid lines 41-44 (see FIG. 4), which in turn are respectively coupled or connected to the blood pressure measuring devices 31-34 (i.e., cuffs). In this way, the blood pressure monitoring device 36 may be used to pneumatically inflate/deflate the cuffs for blood pressure measurements. Also by way of example, the controller 38 may be implemented using appropriate hardware components, such as a processor, memory, etc.

The interface device 37 may further include one or more electrical connectors (e.g., a communications bus plug, such as for a USE bus, measurement probe plug, etc.) for wired connections to the blood pressure measuring devices 31-34. This allows control or measurement data to be communicated to or from the blood pressure measuring devices 31-34. However, in some embodiments, the interface device 37 may in addition, or instead, include a wireless communications circuitry (e.g., cellular, WiFi, Bluetooth, near field communication (NFC), etc.) for wirelessly communicating with the blood pressure measuring devices 31-34. In the case of a wireless blood pressure cuff, the cuff may include a respective pump for inflating the cuff, along with its own wireless communications circuitry for communicating with the interface device 37. In embodiments where disposable cuffs are used, the pump and wireless communications circuit may be enclosed or carried by a housing that may be releasably connected in fluid/electrical communication with the cuff, so that the cuff may be disposed of, but the housing may be readily re-used for another (or the same) patient, for example.

The controller 38 is configured to cooperate with the blood pressure measuring devices 31-34 to determine respective blood pressure measurements for each patient extremity, at Block 53, and selectively determine differences between the blood pressure measurements, at Block 54. It should be noted that differences between all of the blood pressure measurements need not be determined or used in all embodiments. In addition, the controller 38 may also advantageously generate an indication of a probable or potential vascular aneurysm based upon a determined difference between at least two of the blood pressure measurements reaching a vascular aneurysm threshold value, at Blocks 55 and 57, which illustratively concludes the method shown in FIG. 5 (Block 58), although it should be noted that continued blood pressure measurement and monitoring may be performed by the system 30, if desired.

By way of example, a systolic pressure difference of greater than about ten mmHg may be indicative of a vascular aneurysm. More particularly, a threshold range of about twenty to forty mmHg difference (or higher) in systolic pressure may provide a relatively good indication of a potential aneurysm, as will be discussed further below.

A vascular aneurysm is a localized, blood-filled bulge in the wall of a blood vessel, such as an artery or vein. As the size of an aneurysm increases, there is a significant risk of rupture, resulting in severe hemorrhage, other complications, or even death. Acute aneurysms may result in severe localized pain (e.g., in the chest, back, leg, etc.) depending upon their location in the patient's body. However, this may cause first responders (e.g., paramedics, etc.) and even triage providers (e.g., emergency room personnel) to initially misdiagnose the problem. For example, chest pains resulting from a thoracic aneurysm might initially be misdiagnosed as angina or a heart attack. As a result, an emergency care provider may initiate a course of treatment that worsens the aneurysm, such as administering a blood thinner/anticoagulant (e.g., heparin, aspirin, etc.), or sending a patient out for the wrong set of tests/treatment that may prolong the ultimate detection and appropriate treatment for the aneurysm. However, time is typically of the essence in avoiding rupture or other complications from acute aneurysms, and such delays may therefore prove hazardous to the patient.

Yet, the system 30 may advantageously be used by emergency responders or triage providers to quickly and accurately help assess the potential of a vascular aneurysm in a patient. This may potentially help expedite obtaining appropriate follow-up treatment in time to help avoid serious injury or death, and to avoid administering potentially harmful medications to the patient.

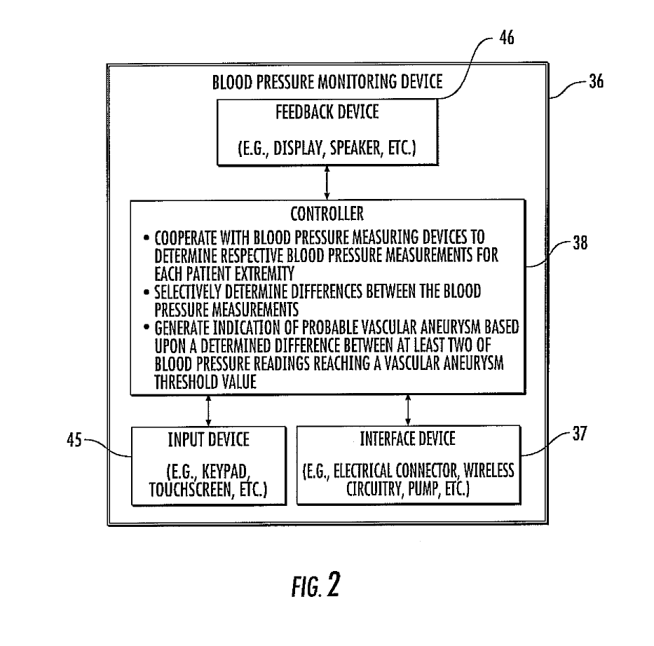

As will be discussed further below, the blood pressure monitoring device 36 may be further configured to advantageously determine a probable vascular aneurysm type from among a plurality of different vascular aneurysm types (e.g., thoracic aortic aneurysm, abdominal aortic aneurysm, peripheral arterial aneurysm, etc.) based upon the respective extremities associated with the blood pressure measurements, at Block 56. More particularly, in the embodiment illustrated in FIG. 2, the blood pressure monitoring device 36 further includes an input device 45 coupled to the controller 38. By way of example, the input device 45 may comprise one or more of a keyboard or keypad, touch screen, menu buttons, a mouse, etc. The input device 45 may advantageously be used by a first responder, etc., to input an indicator of a localized pain region where the patient 35 is experiencing pain (e.g., chest, back, leg, arm, etc.).

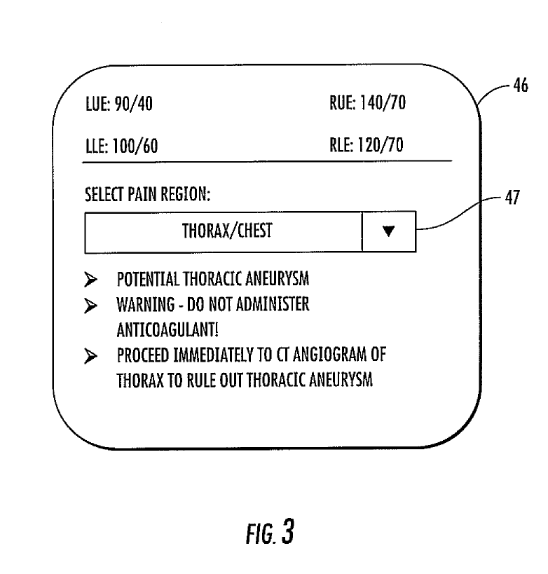

As such, the controller 38 may be further configured to receive the indicator of the localized pain region via the input device 45, and determine the vascular aneurysm type also based upon the localized pain region indicator. That is, by comparing the differences in blood pressure readings in combination with knowing the region where the pain is located, this may allow the controller 38 to provide even more accurate assessments of potential vascular aneurysms. In the example of FIG. 3, in which the feedback device 46 comprises a display, a pull down menu box 47 is provided for selecting the appropriate localized pain region, although other suitable approaches for inputting such data may also be used in different embodiments. For example, symptoms and other patient information may be communicated to the blood pressure monitoring device 36 (e.g., via a wireless communication link, etc.) from a central record keeping system (e.g., a screener initially inputs patient information and symptoms into a patient database upon arrival at ER, etc.). Conversely, symptom information entered directly into the blood pressure monitoring device 36 (e.g., by a paramedic, etc.), as well as measurement and diagnostic data, may be uploaded to the central record keeping system over the air, etc.

The controller 38 may be further configured to determine at least one ankle brachial pressure index (ABI) value based upon the blood pressure measurements (Block 54), and generate the indication of a probable vascular aneurysm (or other vascular condition) also based upon the at least one ABI (Block 55). ABI is the ratio of the blood pressure in the lower legs to the blood pressure in the arms, and is typically calculated by dividing the systolic blood pressure in the leg (e.g., at the ankle) by the systolic blood pressure in the arm. The ABI may also help to further enhance the accuracy of a potential vascular aneurysm determination, as well as to diagnose other vascular problems, as will be discussed further below. In some embodiments, a “cross-body” (i.e., from left arm to right leg, or right arm to left leg) ABI may also be determined and used for diagnostic purposes.

In the example of FIG. 4, the blood pressure monitoring device 36 further includes a housing 60, and a plurality of display windows 61-66 carried by the housing and coupled to the controller 38 (not shown in FIG. 4). The display windows 61-66 are configured to conveniently display RUE blood pressure, LUE blood pressure, RLE blood pressure, LLE blood pressure, right-side ABI, and a left-side ABI, respectively. As such, the blood pressure monitoring device 36 may advantageously provide blood pressure measurements or readings for up to all four patient extremities, as compared to traditional blood pressure measuring devices which require a practitioner to measure blood pressure one extremity at a time.

The input device 45 may advantageously be used to set a particular measurement type, sequence, etc. For example, an option to measure one or more blood pressures at a time may be provided. More particularly, the controller 38 may further be configured to cooperate with the blood pressure measuring devices 31-34 to determine respective blood pressure measurements for one or more patient extremities on a staggered time schedule (e.g., fifteen seconds apart or greater), if desired. That is, the blood pressure measuring devices 31-34 may be actuated at different times, which may provide more accurate readings for determining particular vascular conditions in some circumstances. Moreover, this may be done to cause less stress (i.e., afterload) on the heart, which may be important if a heart attack is a possible or suspect condition (i.e., if a heart attach has not previously been ruled out). Another option is to obtain ABI readings on one or both sides of the patient 35 (or cross-body ABI's). Further, a diagnostic mode may be entered to determine potential vascular aneurysms or other conditions, as discussed above.

In some embodiments, a single blood pressure monitoring device 36 may be used with additional sets of blood pressure measuring devices 31-34 to monitor blood readings and perform diagnoses for more than one patient 35. This may potentially provide several benefits, including reduced storage space requirements, enhanced portability, and reduced cost, for example. While this embodiment may be particularly advantageous where wireless measuring devices 31-34 are used, it may be implemented with “wired” measuring devices as well (or a combination of wired and wireless), in some embodiments.

In this regard, the above-noted features may advantageously be embodied in a non-transitory computer-readable medium having computer-readable instructions for causing a computing device to perform the operations described herein. More particularly, this allows computing devices such as desktop computers, laptop computers, tablet computers, smart phones, personal digital assistants (PDA's), etc. to conveniently interface with the measuring devices 31-34 (e.g., via a cellular, WiFi, Bluetooth, NFC, wired, or other communications link), and receive operator input or provide output through the existing input or output devices thereof, respectively. Thus, by installing an appropriate application or “app” on such devices, they may serve as the blood pressure monitoring device, which not only provides for enhanced convenience of use, it may also result in reduced equipment costs, for example. Moreover, different computing devices may be coupled together in a network, such that one or more central computing devices (e.g., server, desktop or laptop computer, etc.) interface with the measuring devices 31-34, while other portable devices (e.g., tablet computer, smart phone, PDA) obtain feedback data from the central computing device(s).

Referring again to FIG. 3, the controller 38 may be further advantageously configured to selectively output, via the feedback device 46 (i.e., a display in the illustrated example), a warning not to administer a blood thinner or anticoagulant (e.g., aspirin, heparin, etc.) to the patient 35 based upon the determined difference between the at least two of the blood pressure measurements reaching the vascular aneurysm threshold value (i.e., based upon the given likelihood that the patient may in fact have an aneurysm). In the illustrated example, the warning reads “WARNING-DO NOT ADMINISTER ANTICOAGULANT.” In addition, the controller 38 may be similarly configured to cause the display 46 to display a visual indication of the probable vascular aneurysm, which in this example reads “potential thoracic aneurysm.” Further instructions to the care provider are also given in this example, which read “proceed immediately to CT angiogram of thorax to rule out thoracic aneurysm.” The controller 38 is also configured to cause the display 46 to display the blood pressure measurements for each of the extremities, similar to the example of FIG. 4. Here, the extremity blood pressure readings are as follows: LUE =90/40; RUE =140/70; LLE =100/60; and RLE=120/70. Of course, ABI's may similarly be displayed, as described with reference to FIG. 4.

The foregoing will be further understood with reference to several use case scenarios. The first scenario is for a potential thoracic aneurysm, in which the blood pressure readings are those shown in FIG. 3 and set forth above. The patient presents with chest pain or a “ripping” chest pain, and the emergency care provider initiates an immediate blood pressure check for all of the patient's extremities using the system 30. Because the of the difference between the upper and lower systolic blood pressures (right side) is twenty mmHg, an immediate CT (i.e., X-ray computed tomography) angiogram of the thorax is recommended to rule out a thoracic aneurysm. It should be noted that diastolic or mean blood pressure comparisons may also be used in some embodiments. Moreover, cross-body blood pressures may also be compared.

By way of contrast, if the emergency care provider were to use a single-extremity measuring device and only one of the patient's extremities were checked, a misdiagnosis of unstable angina or a potential heart attack, etc., may be reached, and the patient may be sent directly to a heart catheter lab. Generally speaking, blood pressure measurements should be taken on all patient extremities when a patient presents with chest pain or extremity pain. However, because blood pressure measurements are typically performed one extremity at a time in most ER's, urgent care centers, etc., this is very time consuming and may possibly be delayed or even skipped when caregivers are extremely busy or rushed. Moreover, it is common to administer an anticoagulant as a “precaution” when a patient presents with chest pain. Either the delay from the misdiagnosis or the anticoagulant may unfortunately cause the aneurysm to rupture, potentially risking severe injury or death.

In a second use case scenario, where the patient 35 experiences abdominal pain radiating to the back or severe back pain, an immediate blood pressure check of all extremities is performed using the system 30. If there is a blood pressure differential from the upper to the lower extremities of twenty to forty mmHg (e.g., systolic), for example, where the upper pressure is greater than the lower, the blood pressure monitoring device 36 may advantageously diagnose a potential abdominal aortic aneurysm, and recommend an abdominal aortic ultrasound or CT angiogram of the abdominal aortic to rule out an abdominal aortic aneurysm.

In accordance with a third use case scenario pertaining to a suspect peripheral aneurysm, the patient 35 presents with severe leg pain, numbness, or cramping. An immediate blood pressure check of all extremities is again performed using the system 30. On the unilateral (i.e., same) side, an upper blood pressure that is greater than the corresponding lower blood pressure by twenty mmHg or more may be indicative of a femoral or popliteal artery aneurysm. Here, the course of action provided by the blood pressure monitoring device 36 may be to obtain an immediate arterial Doppler scan of the affected lower extremity, and not to administer an anticoagulant.

In a fourth use case scenario pertaining to a peripheral arterial vascular disease (PAD), the patient 35 presents with severe leg pain, cold feet, or a pain with exertion (i.e., exercise). Generally speaking, exemplary ranges for PAD classification may be 0.8-1 for mild disease, 0.5-0.8 for moderate disease, and 0.0-0.5 for severe disease. As such, if the blood pressure monitoring device 36 determines an ABI of less than 0.6, this may be used as the threshold or trigger point for a diagnosis of potential claudication. Accordingly, the blood pressure monitoring device 36 may recommend obtaining an arterial Doppler scan of the affected leg, and may note that there is a low risk associated with administering an anticoagulant (provided that the patient is not allergic to aspirin, heparin, etc.).

In addition to the foregoing examples, the blood pressure monitoring device 36 may also provide potential diagnoses and a recommended treatment or course of action for other vascular conditions. By way of example, such conditions may include the following: dissecting thoracic aneurysm; coarctation of the aorta; tetralogy of fallot; transposition of the great vessels; subclavian artery stenosis; subclavian steal syndrome; Marfan's syndrome; common iliac artery stenosis; external iliac artery stenosis; auxiliary artery stenosis; congenital diseases; aortic dissections; acute coronary syndromes; chest trauma from a motor vehicle accident or other blunt chest wall trauma; etc. In such instances, measured blood pressure and/or ABI values, patient symptoms, etc., may be used by the controller 38 to determine a potential diagnosis for these various conditions based upon appropriate respective blood pressure and/or ABI suspect ranges, as will be appreciated by those skilled in the art, similar to the examples provided above.

The system 30 may therefore provide enhanced blood pressure monitoring and diagnostic features in numerous clinical settings, including primary care, urgent care, pediatrics, cardiology, ER/hospitals, first responders (e.g., paramedics), etc. In the case of first responders, the system 30 may be of particular advantage because it may help prevent administering of an anticoagulant when this may be detrimental, and also to provide advance notice to a hospital, ER, etc., of what test facilities need to be made immediately available for the patient upon arrival (e.g., ultrasound, CT, etc.). In an example pediatric application, coarctation of the aorta is a congenital condition in which the aorta narrows in the area of the ductus arteriosus, typically resulting in diminished blood pressure in the LUE. However, due to time constraints, etc., a care provider may typically only subjectively palpate for blood pressure differences. Yet, use of the blood pressure monitoring system 30 may advantageously allow for a more accurate assessment or diagnosis of this potential condition (or others) in newborns or children (along with a corresponding recommendation of the appropriate follow-up testing or procedures), which might otherwise be missed by subjective palpation alone.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims. that which is claimed is:

1. A blood pressure monitoring system comprising:
a plurality of a blood pressure measuring devices each configured to measure blood pressure of a respective extremity of a patient; and
a blood pressure monitoring device comprising at least one interface device configured to interface with said plurality of blood pressure measuring devices, and
a controller coupled to said at least one interface device and configured to
cooperate with said plurality of blood pressure measuring devices to determine respective blood pressure measurements for each patient extremity,
selectively determine differences between the blood pressure measurements, and
generate an indication of a probable vascular aneurism based upon a determined difference between at least two of the blood pressure measurements reaching a vascular aneurysm threshold value.

2. The blood pressure monitoring system of claim 1 wherein said blood pressure monitoring device is further configured to determine a probable vascular aneurism type from among a plurality of different vascular aneurism types based upon the extremities associated with the at least two blood pressure measurements.

3. The blood pressure monitoring system of claim 2 wherein said blood pressure monitoring device further comprises an input device coupled to said controller; and wherein said controller is further configured to receive an indicator of a localized pain region via said input device, and determine the vascular aneurism type also based upon the localized pain region indicator.

4. The blood pressure monitoring system of claim 1 wherein said controller is configured to cooperate with said plurality of blood pressure measuring devices to determine respective blood pressure measurements for each patient extremity on a staggered time schedule.

5. The blood pressure monitoring system of claim 1 wherein said controller is further configured to determine at least one ankle brachial pressure index (ABI) value based upon the blood pressure measurements, and generate the indication of a probable vascular aneurism also based upon the at least one ABI.

6. The blood pressure monitoring system of claim 1 wherein said blood pressure monitoring device further comprises at least one feedback device coupled to said controller; and wherein said controller is further configured to selectively output, via said at least one feedback device, a warning not to administer an anticoagulant to the patient based upon the determined difference between the at least two of the blood pressure measurements reaching the vascular aneurism threshold value.

7. The blood pressure monitoring system of claim 1 wherein the vascular aneurism threshold comprises at least a twenty mmHg systolic pressure difference.

8. The blood pressure monitoring system of claim 1 wherein said blood pressure monitoring device further comprises at least one display coupled to said controller; and wherein said controller is further configured to cause said at least one display to display a visual indication of the probable vascular aneurism.

9. The blood pressure monitoring system of claim 9 wherein said controller is further configured to cause said at least one display to display the blood pressure measurements for each of the extremities.

10. The blood pressure monitoring system of claim 1 wherein at least one of said blood pressure measuring devices comprises a wireless blood pressure measuring device; and wherein said at least one interface device comprises a wireless communications circuit.

11. A blood pressure monitoring device for use with a plurality of a blood pressure measuring devices each configured to measure blood pressure of a respective extremity of a patient, the blood pressure monitoring device comprising:
at least one interface device configured to interface with said plurality of blood pressure measuring devices; and
a controller coupled to said at least one interface device and configured to
cooperate with the plurality of blood pressure measuring devices to determine respective blood pressure measurements for each patient extremity,
selectively determine differences between the blood pressure measurements, and
generate an indication of a probable vascular aneurism based upon a determined difference between at least two of the blood pressure measurements reaching a vascular aneurism threshold value.

12. The blood pressure monitoring device of claim 11 wherein said controller is further configured to determine a probable vascular aneurism type from among a plurality of different vascular aneurism types based upon the extremities associated with the at least two blood pressure measurements.

13. The blood pressure monitoring device of claim 12 further comprising an input device; and wherein said controller is further configured to receive an indicator of a localized pain region via said input device, and determine the vascular aneurism type also based upon the localized pain region indicator.

14. The blood pressure monitoring device of claim 11 wherein said controller is configured to cooperate with the plurality of blood pressure measuring devices to determine respective blood pressure measurements for each patient extremity on a staggered time schedule.

15. The blood pressure monitoring device of claim 11 wherein said controller is further configured to determine at least one ankle brachial pressure index (ABI) value based upon the blood pressure measurements, and cause said at least one feedback device to output the indication of a probable vascular aneurism also based upon the at least one ABI.

16. The blood pressure monitoring device of claim 11 further comprising at least one feedback device coupled to said controller; and wherein said controller is further configured to selectively output, via said at least one feedback device, a warning not to administer an anticoagulant to the patient based upon the determined difference between at least two of the blood pressure measurements reaching the vascular aneurism threshold value.

17. A blood pressure monitoring method comprising:
coupling a plurality of a blood pressure measuring devices each to a respective extremity of a patient;
using a blood pressure monitoring device to
cooperate with the plurality of blood pressure measuring devices to determine respective blood pressure measurements for each patient extremity,
selectively determine differences between the blood pressure measurements, and
generate an indication of a probable vascular aneurism based upon a determined difference between at least two of the blood pressure measurements reaching a vascular aneurism threshold value.

18. The method of claim 17 further comprising using the blood pressure monitoring device to determine a probable vascular aneurism type from among a plurality of different vascular aneurism types based upon the extremities associated with the at least two blood pressure measurements.

19. The method of claim 18 further comprising using the blood pressure monitoring device to receive an indicator of a localized pain region via an input device, and determine the vascular aneurism type also based upon the localized pain region indicator.

20. The method of claim 17 wherein the respective blood pressure measurements for each patient extremity are measured on a staggered time schedule.

21. The method of claim 17 further comprising using the blood pressure monitoring device to determine at least one ankle brachial pressure index (ABI) value based upon the blood pressure measurements, and to cause at least one feedback device to output the indication of a probable vascular aneurism also based upon the at least one ABI.

22. The method of claim 17 further comprising using the blood pressure monitoring device to selectively output, via at least one feedback device, a warning not to administer an anticoagulant to the patient based upon the determined difference between the at least two of the blood pressure measurements reaching the vascular aneurism threshold value.

23. A non-transitory computer-readable medium having computer-executable instructions for causing a computing device to perform steps comprising:
cooperating with a plurality of blood pressure measuring devices each coupled to a respective extremity of a patient to determine respective blood pressure measurements for each patient extremity;
selectively determining differences between the blood pressure measurements; and
generating an indication of a probable vascular aneurysm based upon a determined difference between at least two of the blood pressure measurements reaching a vascular aneurysm threshold value.

24. The non-transitory computer-readable medium of claim 23 further having computer-executable instructions for causing the computing device to perform a step comprising determining a probable vascular aneurysm type from among a plurality of different vascular aneurysm types based upon the extremities associated with the at least two blood pressure measurements.

25. The non-transitory computer-readable medium of claim 24 wherein the computing device comprises an input device; and further having computer-executable instructions for causing the computing device to perform a step comprising receiving an indicator of a localized pain region via the input device; and wherein determining the vascular aneurysm type further comprises determining the vascular aneurysm type also based upon the localized pain region indicator.

26. The non-transitory computer-readable medium of claim 23 wherein cooperating further comprises causing the blood pressure measuring device to perform respective blood pressure measurements for each patient extremity on a staggered time schedule.

27. The non-transitory computer-readable medium of claim 23 further having computer-executable instructions for causing the computing device to perform a step comprising determining at least one ankle brachial pressure index (ABI) value based upon the blood pressure measurements; and wherein generating further comprises generating the indication of a probable vascular aneurysm also based upon the at least one ABI.

28. The non-transitory computer-readable medium of claim 23 wherein the computing device further comprises at least one feedback device; and further having computer-executable instructions for causing the computing device to perform a step comprising selectively outputting, via the at least one feedback device, a warning not to administer an anticoagulant to the patient based upon the determined difference between the at least two of the blood pressure measurements reaching the vascular aneurysm threshold value.
